(19) United States
(12) Patent Application Publication
(10) Pub. No.: US 2024/0374403 A1
(43) Pub. Date: Nov. 14, 2024

(54) ORTHOPAEDIC DEVICE

(71) Applicant: OTTOBOCK SE & CO. KGAA, Duderstadt (DE)

(72) Inventors: Jonas BORNMANN, Duderstadt (DE); Mario KOPPE, Duderstadt (DE); Randolph MAUßNER, Duderstadt (DE); Lars Benjamin FINKE, Duderstadt (DE)

(21) Appl. No.: 18/687,526

(22) PCT Filed: Aug. 30, 2022
(86) PCT No.: PCT/EP2022/074066

§ 371 Date: Feb. 28, 2024

(30) Foreign Application Priority Data
Sep. 1, 2021 (DE) 10 2021 122 608.2

(51) Int. Cl. A61F 2/80; A61F 2/68; A61F 2/70

(52) U.S. Cl. CPC A61F 2/80; A61F 2002/6827; A61F 2002/701

(57) ABSTRACT

The invention relates to an orthopaedic device having at least one wall which at least partly surrounds a stump or appendage when applied; —the wall has a variable inner periphery and forms an entry opening; —the wall is assigned an actuator, mounted on the orthopaedic device, for at least one actuating element which is mounted on the orthopaedic device and can be used to vary the inner periphery of the wall, wherein at least one sensor device is assigned to the mounting of the actuator and/or the actuating element in order to determine mounting forces of the actuator and/or the actuating element.

The invention relates to an orthopaedic device having at least one wall which at least partially surrounds a stump or limb when fitted, which wall has a variable inner periphery and forms an entry opening, wherein the wall is assigned an actuator, mounted on the orthopaedic device, for at least one actuation element which is mounted on the orthopaedic device and which can be used to vary the inner periphery of the wall. The invention also relates to a method for controlling and adapting an inner periphery of a wall of an orthopaedic device.

Orthopaedic devices are technical components which are worn on the body of a person for medical reasons, in particular prostheses, orthoses and, as a special case, exoskeletons.

Prostheses serve for replacing non-existent or no longer existent limbs. Prostheses are intended not only to approximate the external form of the limbs that are to be replaced, but also to as far as possible completely replicate said limbs in terms of their function. Aside from purely mechanical prostheses, numerous electronically controlled prostheses also exist. The control relates for example to the adaptation of prosthetic devices to different usage conditions or usage requirements. Prostheses commonly have more than one component, which components can be adjusted or displaced relative to one another. For example, prosthetic joints are provided for the pivoting of an upper part relative to a lower part, with a resistance device or a drive being arranged between the upper part and the lower part, which resistance device or drive can be varied on the basis of sensor data that are evaluated in a control device. An actuator, for example a motor or some other adjustment device or drive, may adjust valves in order to vary resistances to movement. An electric drive may be switched into a generator mode in order to provide resistance to a pivoting movement, or the drive may alternatively be activated in order to perform or assist a movement. A magnetic field may be generated, for example by means of an electromagnet, in order to vary viscosity characteristics of a magnetorheological medium.

Prostheses are fixed to the body of the patient, and there are various technologies for this purpose. In the vast majority of cases, the fixing is performed by way of a prosthesis socket which is of cup-like design and has a proximal entry opening that extends around the stump. The socket is dimensionally stable and, at its distal end, has further prosthetic devices, for example a prosthetic joint or an end component such as a prosthetic foot or a prosthetic hand.

Aside from an individual adaptation of the prosthesis socket and fixing for example by means of liner technology using a so-called pin lock, prosthesis sockets also exist which have multiple struts or wall parts that are displaceable relative to one another.

DE 10 2010 019 843 A1 relates to a prosthesis socket having a distal end piece, having securing devices for a prosthetic knee joint, having a radially braceable sleeve, and having a tensioning device for adapting a receiving space of the sleeve to the stump. The sleeve may have at least two segments which are connected to a supporting frame and which partially overlap one another. The tensioning device has at least one cable pull which extends across the segments and which can be adjusted in terms of an effective length by means of an adjusting device.

US 2010/274364 A1 relates to a prosthesis socket having at least one opening in which in each case one plate is arranged, said plate being pushed through the opening and against an amputation stump, which is received by the prosthesis socket, by means of tensioning devices that have actuators and a control device. At least one pressure sensor is arranged in a lower portion of one of the plates.

WO 2014/144985 A1 relates to a prosthetic device having a prosthesis socket and having a sensor arrangement in order to sense the ambulatory state and forces and/or pressures that are applied to the received stump by the prosthetic device. The fit of the prosthesis socket and of the prosthetic arrangement on the stump is adapted and varied by means of hydraulic actuators that are controlled in accordance with the detected parameters.

US 2010/0274364 A1 relates to a prosthesis having a prosthesis socket in which a window is formed, within which window an adjustable panel is movably arranged. A receiving space for a limb stump is formed by the prosthesis socket and the adjustable panel. Using a traction mechanism, the position of the adjustable panel and thus the receiving volume can be varied by adjustment of a tensile force that is exerted on the traction mechanism. The adjustment of the tensile force is performed on the basis of sensor data from at least one sensor.

EP 3 454 792 B1 relates to a prosthesis socket having a proximal introduction opening, having an inner periphery which at least partially surrounds a stump, having at least one connection device for a prosthesis component that can be secured to the prosthesis socket, and having at least one actuator by means of which the inner periphery of the prosthesis socket can be varied. At least one sensor is coupled to a control device, with said sensor being designed as an inertial sensor. The control device is connected to the actuator and activates same in accordance with the sensor signals received from the inertial sensor.

WO 2018/017959 A1 relates to a system and a method for detecting the distribution of forces that are transmitted from a body and a limb stump to a prosthesis socket. A multiplicity of sensors is arranged on the prosthesis socket, said sensors covering a multiplicity of inner regions. A processor is coupled to the network of sensors and receives sensor data, on the basis of which the pressure distribution is ascertained.

WO 2014/138 297 A1 relates to methods and devices for the automatic closure of medical devices or apparatuses, in particular prosthesis sockets or orthoses, having a tensioning system that is coupled to a drive. On the basis of sensor values such as the pressure between a socket wall and a limb, or on the basis of tensile forces within a tensioning strap, settings are automatically varied and the tensioning system is tightened or loosened.

Orthoses are orthopaedic aids which are fitted onto an existing limb and which perform, restrict or assist movements. Drives or resistance devices may be arranged between articulatedly interconnected components, which drives or resistance devices can be adjusted or set correspondingly to devices on prostheses. Here, too, the adjustment is performed on the basis of sensor data that are transmitted to a data processing device. The sensor data and the adjustment commands may be transmitted wirelessly to the actuator. In the context of this application, orthoses are also understood to include exoskeletons which are fitted onto the body of a patient and which form an external supporting structure, in particular for the purposes of guiding and influencing the movements of a user, for example assisting such movements by means of drives or braking such movements by means of resistance devices. Orthoses, and exoskeletons as special cases, may be used not only for assisting everyday activities but also for training purposes or therapeutic purposes.

Current prostheses or orthoses with sensor-based adjustment devices or actuators are delivered with software that is adapted and configured for the particular patient. During the configuration, individual parameters may be varied, for example in order to adapt the damping behavior of a resistance device to the particular user. It is likewise possible for individual functions to be activated or deactivated, for example because the patient in question cannot or should not perform said function. Orthoses are fixed using securing elements that are arranged on rails or struts. The rails or struts are generally articulatedly connected to one another or resiliently mounted on one another. For the purposes of securing and firmly fitting the orthoses onto the respective limbs or the torso, shells or braces are provided which at least partially surround limbs.

WO 2013/191933 A2 relates to an orthosis with a treatment regimen. The orthosis, in particular lumbar orthosis, has a tensioning device for a tensioning means and has a motor drive. The tensioning device displaces two orthosis segments toward one another, or loosens the tensioning device such that the orthosis segments can be moved apart by the application of a restoring force. The orthosis segments are coupled to one another in the abdomen region by means of a hook and loop fastener, and the tensioning device, which automatically tightens or loosens the orthosis, is arranged in the region of the back. Pressure sensors are arranged in the orthosis in order to measure the tension. Also, if changes in the position of the user are detected, the tension of the tensioning device is automatically adapted. A massage function can be provided by way of periodic tightening and loosening of the tensioning device.

US 2014/0068838 A1 relates to a motorized tensioning system for shoes, posture correction devices, backpacks, head coverings or orthoses. The motor sets the desired tension by way of a tensioning means, for example a cable or a cord. The tension can be adjusted by remote control.

An adjustment of the tension in the particular tensioning system is performed on the basis of direct pressure measurements between the wall and the body part or by way of tension measurements within the traction mechanism. In the case of a direct pressure measurement, the very great measurement deviations that result from contact with soft tissue, such as arises depending on the load or on changes that occur whilst the item in question is being worn, pose a problem. In the case of forces being measured within the tensioning means, the location of the measurement, and the high forces acting there, pose a problem.

It is an object of the present invention to provide an orthopaedic device and a method for adapting an inner periphery of a wall of such an orthopaedic device, with which device and method the above-described problems can be avoided or at least alleviated.

The object is achieved by means of an orthopaedic device having the features of the main claim and a method having the features of the independent claim. Advantageous embodiments and refinements of the invention are disclosed in the subclaims, in the description and in the figures.

The orthopaedic device having at least one wall which at least partially surrounds a stump or limb when the orthopaedic device has been fitted, which wall has a variable inner periphery and forms an entry opening, wherein the wall is assigned an actuator, mounted on the orthopaedic device, for at least one actuation element which is mounted on the orthopaedic device and which can be used to vary the inner periphery of the wall, provides for at least one sensor device to be assigned to the mounting of the actuator and/or of the actuation element in order to determine mounting forces of the actuator and/or of the actuation element. By virtue of the mounting forces of the actuator and/or of the actuation element being detected by means of sensors, or by virtue of the mounting forces of the actuator and/or of the actuation element being determined using the sensor data detected by the sensor device, it is possible, without directly measuring pressure forces, to draw conclusions regarding the pressure conditions within a socket or within a wall of an orthopaedic device. Instead of performing a direct measurement of radial forces, an indirect measurement of easily and reproducibly detectable variables can be used to infer the pressure conditions within the wall. It is thus possible to achieve an exact and reliable mechatronic adaptation of the wall to the body part, the limb or the stump. Furthermore, the sensors do not make direct contact with the skin or any soft structure, for example a liner, such that reproducible results are easy to achieve. The possibility of detecting indirect measurement variables allows positioning and arrangement on the wall or in the wall at locations, and in a manner, which effectively protect(s) the sensors or the sensor device against external influences. The bearing force of the actuation element for example on the outer side of the wall in a guide can be detected, with regard to the force exerted on the wall, by means of a sensor, and said bearing force is part of the mounting forces that act on the actuation element.

In one refinement, the actuator, the actuation element and/or a diverting device that is assigned to the actuation element is mounted in floating fashion on the orthopaedic device, in particular on the wall. A floating mounting is in particular a displaceable, tiltable or rotatable mounting. Depending on the load on the actuator, on the actuation element or on the diverting device for the actuation element, for example the mounting point for a loop, an eyelet, a roller or a diverting pin, said components are moved in a load-dependent manner owing to the floating mounting. This movement is detected by means of the sensor device and evaluated. For example, if the actuator is mounted in a guide so as to be longitudinally displaceable relative to a pressure sensor, the slight displacement relative to the pressure sensor is measured and is taken into consideration for the determination of the pressure conditions within the wall. The determination of the pressure conditions is performed on the basis of the sensor values in a control device that is equipped with the necessary components. These include not only a processor for processing sensor signals but also further data processing devices, memories, wired and/or wireless interfaces, software and energy stores.

In one embodiment, the sensor device has at least one sensor that detects distances, forces and/or moments. The sensor may be a capacitive sensor, a resistive sensor, an inductive displacement transducer or inductive spacing sensor, or may be based on optical operating principles. For example, displacements or deformations at mounting points or securing elements may be detected by means of strain gauges or pressure-sensitive sensors, piezo elements or the like. Distances may be detected by means of Hall sensors or optical sensors, in the case of which changes in position between components are detected and are transmitted to an evaluation and/or control device. The mounting forces are advantageously ascertained directly, in particular at the location at which the actuator and/or the actuation element is mounted on the wall. Increased computational effort is thus avoided, and the directly acting forces can be detected. The mounting forces or the forces that act on the mounting of the actuator or of the actuation element can thus be directly and quickly detected and determined. The force on the bearing arrangement can be detected in real time over the entire service life of the orthopaedic device without the need for external energy to be supplied to the actuator. The mounting forces are detected irrespective of the operating state of the actuator, and, in particular if the actuator is designed as a motor, irrespective of whether or not the motor is actuated. For the sensor arrangement that is independent of the actuator and of the operating state thereof, energy from the battery or a storage battery may be used.

In one embodiment, the sensor device is designed or arranged to detect forces acting in a proximal-distal direction, in a radial direction and/or in a peripheral direction of the wall. It is thus possible to record all force directions in the orthopaedic device, in particular in a prosthesis socket or an orthosis component, and detect the corresponding mounting forces of the actuator and/or of the actuation element. In one embodiment, the sensor device or the sensors are positioned separately from the actuator at the mounting sites of the actuator and/or of the actuation element and continuously detect the acting bearing forces in real time.

In one embodiment, the actuation element is designed as a flexible traction element, in particular as a strap, as a cord or as a cable, or as a combination of these. With a flexible, in particular non-elastic traction element, it is easily possible for forces to be transmitted and transferred from the actuator to the orthopaedic device, and in particular for forces acting in a peripheral direction to be applied.

In one embodiment of the invention, the actuator has a slide, a spindle or a roller, which slide, spindle or roller is connected to the actuation element. It is thus possible for the forces that are applied by means of the actuator or the actuation element to be redirected, and for a corresponding displacement, tensioning action or movement to be effected on or within the orthopaedic device.

The wall may be formed in multiple parts or divided into segments that are displaceable relative to one another, in order to achieve an adjustment or setting of the orthopaedic device by means of the actuator and the actuation element.

In one embodiment, the actuator is driven by motor means, though the actuator may also be driven manually, for example by way of a spindle, an adjustable wheel, a turn-lock fastener, a ball, a lever or some other actuating device. Activation or drive by motor means is performed in particular by means of an electric motor; other drives such as linear drives are likewise provided for driving the actuator.

In one embodiment, the sensor device is connected to a control device which activates and/or deactivates a motor drive of the actuator on the basis of sensor values from the sensor device and/or transmits a display or output command to a display or output device. Automatic setting or adaptation is thus possible on the basis of sensor values, and/or a corresponding warning or item of information can be transmitted to the user or an orthopaedic technician or some other person or institution.

The orthopaedic device is designed in particular as a prosthesis socket, an orthosis or an exoskeleton.

The method for controlling an adaptation of an inner periphery of a wall of an orthopaedic device, in particular of a prosthesis socket or an orthosis or an exoskeleton as described above, provides for sensor values to be detected by the sensor device, and for a drive of the actuator to be activated or deactivated if set threshold values are overshot and/or undershot. It is thus possible, on the basis of sensor values, to perform an automatic adaptation of an inner periphery of a wall, and to prevent an orthopaedic device from lying too tightly or too loosely against the particular user.

In one refinement of the method, different threshold values are defined for different usage situations, wherein the particular usage situation is automatically identified on the basis of sensor values or is manually selected. The sensors or the sensor device can for example identify the present movement state and/or load state of the orthopaedic device. For example, if high acceleration forces act on the orthopaedic device and these are assigned, by way of the sensor values, to an actuation, the inner periphery of the orthopaedic device may be reduced in order to ensure that the orthopaedic device lies tightly against the user. If no movement data are detected by means of the sensor device, this indicates, for example, that the user is sitting or lying down, such that the inner periphery of the wall is enlarged in order to improve blood flow and relieve tension on the soft tissue. Alternatively, the particular movement situation or load situation is set manually, for example by means of an interface that is coupled by wire or wirelessly to the orthopaedic device.

The sensor values are advantageously ascertained at different points on the orthopaedic device, for example in order to precisely detect movement situations or identify the load situation and thus also the fit of the orthopaedic device on the user.

In one embodiment, the mounting forces of the actuator and/or of the actuation element are detected independently of the operating state of the actuator, in particular in real time and over the entire service life of the orthopaedic device. Owing to the fact that the mounting forces are measured directly irrespective of the operating state of the actuator, it is not necessary for the actuator, in an embodiment as a motor, to be electrically energized or actuated in order to detect sensor data, measure characteristic variables and from these either directly measure or calculate the mounting forces.

Exemplary embodiments of the invention will be discussed in more detail below on the basis of the figures. In the figures:

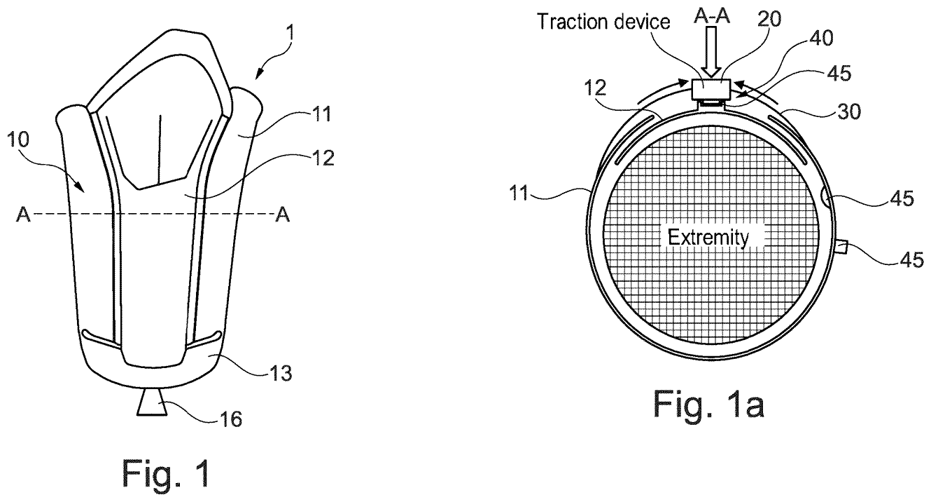

FIG. 1—shows an orthopaedic device in the form of a prosthesis socket;

FIG. 1a—is a sectional illustration through an orthopaedic device with an actuator;

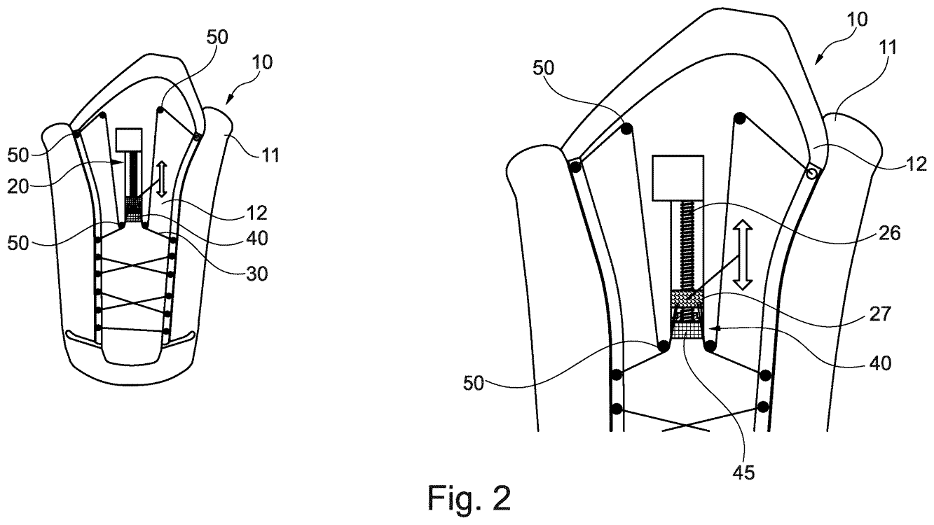

FIG. 2—shows two views of a variant of FIG. 1a;

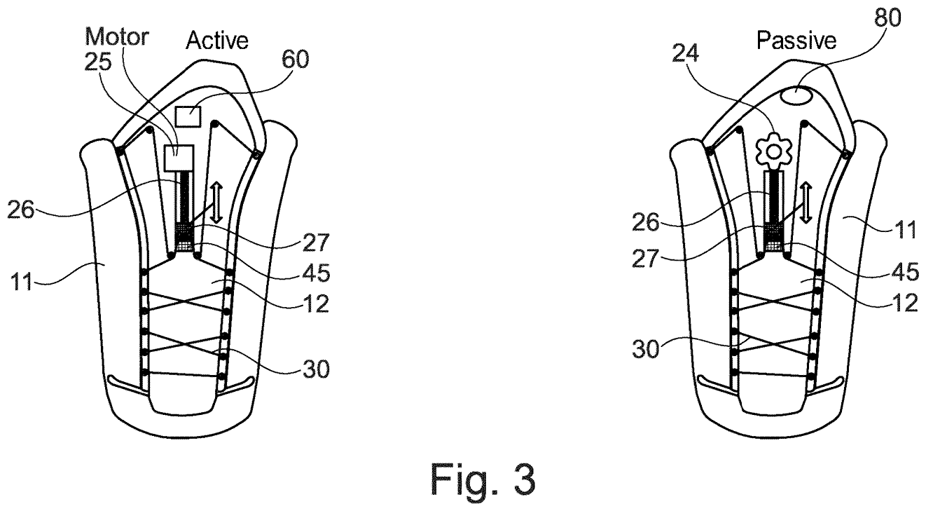

FIG. 3—shows variants of FIG. 2 with motor-based and manual adjustment;

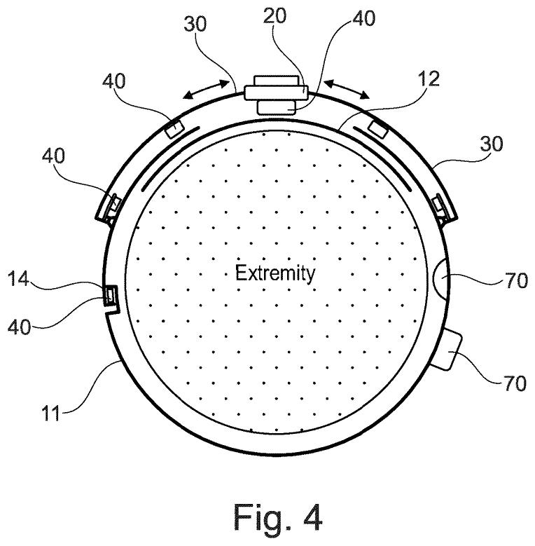

FIG. 4—is a sectional illustration through a variant of FIG. 1a;

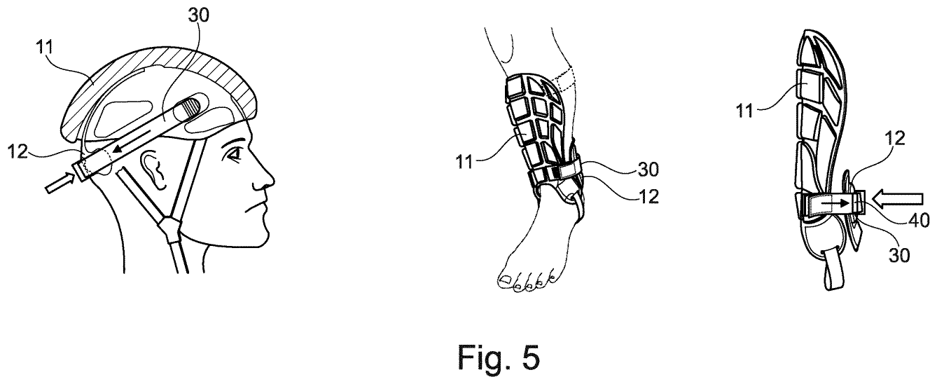

FIG. 5—shows exemplary embodiments;

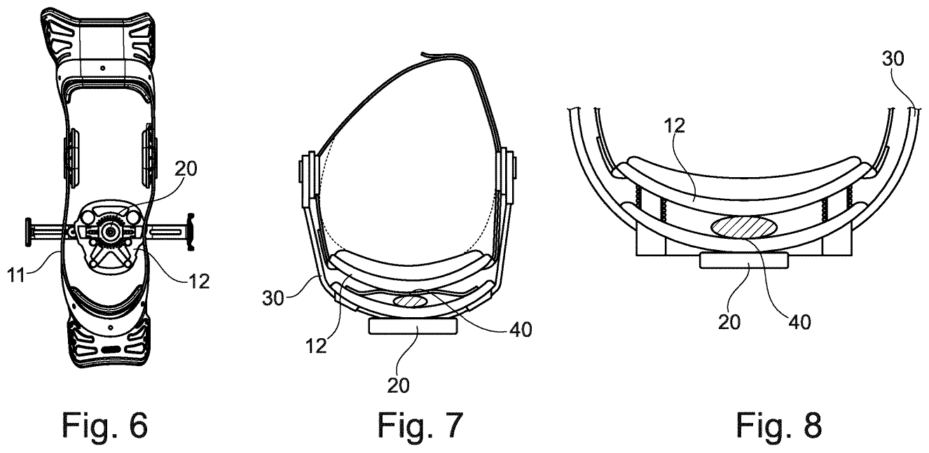

FIG. 6—shows an application in the case of a knee orthosis;

FIG. 7—shows a sectional illustration through a knee orthosis;

FIG. 8—shows a variant of FIG. 7; and

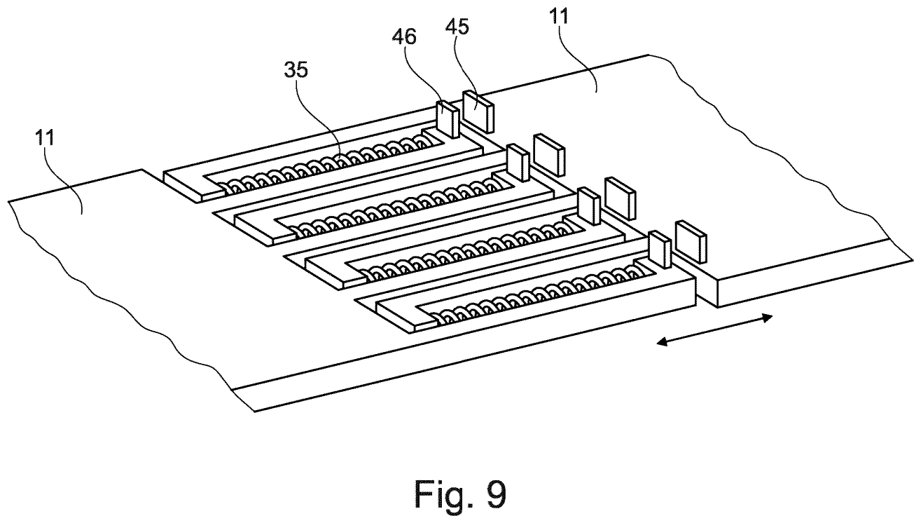

FIG. 9—shows an exemplary embodiment of a sensor.

FIG. 1 shows an orthopaedic device 1 in the form of a prosthesis socket in a schematic, perspective illustration. The prosthesis socket has a proximal entry opening and, when said prosthesis socket has been fitted, the wall 10 of said prosthesis socket completely surrounds a stump. The wall 10 consists of multiple segments 11, 12, 13, wherein the first segment 11 is formed as a single piece over a major part of the periphery and leads into a distal end segment 13. By means of slots, which are arranged in a peripheral direction, between the end segment 13 and the first segment 11 in the distal portion of the prosthesis socket, the two segments 11, 13 can be displaced relative to one another. A third, separate segment 12 is arranged on the orthopaedic device 1 and, in the exemplary embodiment illustrated, closes the gap in the periphery of the first segment 11. A connection device 16 in the form of a pyramid-shaped adapter for securing further prosthetic components, for example a prosthetic knee joint and/or a lower leg tube, is arranged or formed on the distal end of the prosthesis socket 1.

FIG. 1a shows, in a sectional illustration according to the line A-A in FIG. 1, a modified orthopaedic device 1 in which it is possible to see the arrangement of the third segment 12 behind the gap between the two mutually opposite edges, running in a proximal-distal direction, of the first segment 11. The third segment 12 is displaceable relative to parts of the first segment 11 and may for example be secured or arranged on the end segment 13 (not shown). Furthermore, an actuator 20 for actuating an actuation element 30, by means of which the inner periphery of the wall 10 can be varied, is arranged on the third segment 12 as part of the wall 10. The actuation element 30 is in particular in the form of a flexible, optionally tension-resistant or elastic component, for example a cord, belt, cable or the like, and in the exemplary embodiment illustrated is secured to mutually opposite regions of the first segment 11 of the wall 10. If the actuation element 30 is varied in terms of its effective length, for example rolled up, by means of the actuator 20, or if those ends or regions of the actuation element 30 which are fixed to opposite portions of the wall 10 are moved toward one another, the inner periphery of the orthopaedic device or of the wall 10 varies, because the two mutually opposite edges of the gap in the wall 10 are moved toward one another. Instead of opposite ends of the actuation element 30 being secured to the wall 10, it is also possible for the inner periphery to be varied by shortening the effective length of a loop that is formed by the actuation element 30. Here, diverting points on which the actuation element 30 is guided are displaced toward one another or moved out of their initial position in order to vary the inner periphery of the cavity that is at least partially surrounded by the wall 10.

The actuation element 30 is secured to mutually opposite portions of the first segment 11 of the wall 10, and is equipped with a sensor 45 there. A sensor device 40 is arranged at the mounting of the actuation element 30 on the third segment 12, which sensor device detects mounting forces of the actuation element 30 relative to the third segment 12. The sensor device 40 has at least one sensor 45 which detects distances, spacings, forces and/or moments, in particular the forces and/or moments that act directly on the mounting points. The detection may be performed by optical or capacitive means, by measuring resistances, by means of strain gauges or piezo elements, by inductive means, or in some other way. In particular, the sensor device 40 may have a sensor 45 which is designed as a Hall sensor or which has a piezo element. Mounting forces of the actuation element 30 on the wall 10 are detected at the inner periphery or the outer periphery of the wall 10 by means of the sensors 45 or by means of at least one sensor 45. The other sensor may be used to detect further characteristic variables or operating parameters, for example to detect temperature, moisture, pressures, accelerations, positions in space, angles or the like.

In the exemplary embodiment illustrated, the actuation element 30 is assigned an actuator 20 that is used to move the actuation element 30. The actuator 20 is designed for example as a motor or setting wheel that is used to vary the effective length of the actuation element 30. The actuator 20 is supported relative to the third segment 12 at a mounting point, wherein at least one sensor 45 of the sensor device 40 is situated between the mounting point and the actuator 20 in order to detect mounting forces of the actuator 20 and/or of the actuation element 30 that is connected to the actuator 20. Here, the mounting forces may take the form of radially acting pressure forces, tension forces or pressure forces acting in a peripheral direction, or torques. The forces or moments may be measured directly by means of pressure sensors, force sensors or torque sensors, or by indirect measurement of deformations or changes in length, for example. Here, the forces or moments are measured irrespective of the operating state of the actuator and of the orthopaedic device. The measurement is advantageously performed continuously during the use of the orthopaedic device. The measurement is performed in real time, wherein the evaluation of the measured values or sensor values may be performed cyclically.

The extremity, in particular a stump in the case of a prosthesis socket, is arranged within the orthopaedic device and is enclosed by the wall. In the example illustrated, the segments 11, 12 overlap in a peripheral direction, such that the resulting wall extends over the entire periphery of the extremity. It is alternatively possible for a gap to exist between two segments 11, 12, which gap is varied by virtue of the actuation element 30 being actuated.

FIG. 2 illustrates a variant of the embodiment according to FIG. 1, in which it is likewise the case that multiple segments 11, 12 of the wall 10 are provided. Furthermore, multiple actuation elements 30 are provided, specifically a proximal actuation element 30 and a distal actuation element 30, which are formed separately and are fixed to different regions of the wall 10. Both actuation elements 30 are designed as cables, cords or wires, and are guided on diverting devices 50 which are arranged or formed both on the third segment 12 and on the first segment 11. The distal actuation element 30 is guided in criss-crossing fashion as a loop in the diverting devices 50, similarly to the laces on a shoe, with two ends of the distal actuation element 30 being secured to a movable slide 27 or carriage which is guided in a guide and which can be displaced in one or the other direction by means of a spindle 26. The proximal actuation element 30 is fixed, at proximal ends, in the region of the mutually opposite edges of the first segment 11, and is guided inward and downward by means of diverting elements 50 and is likewise secured to the slide 27 or carriage. In the exemplary embodiment illustrated, the spindle 26 is oriented in a proximal-distal direction such that, when the spindle 26 rotates in one direction, the slide 27 is displaced upward and the effective length between the respective diverting elements 50 or securing points is shortened. The mutually opposite edges of the gap within the wall 10 are thus moved toward one another, and the inner periphery of the wall 10 is reduced. In the case of a movement in the reverse direction, the slide 27 moves downward, and each actuation element 30 is relieved of tension and the inner periphery of the wall 10 increases in size or can be increased in size. If the wall 10 exhibits elasticity, the inner periphery will increase in size of its own accord owing to the restoring forces. The actuator 20 may have a motor or a manually actuated drive device. The spindle 11 or the slide 27 is supported relative to the third segment 12 on the sensor device 40, which has at least one sensor 45 arranged therein or thereon. The sensor 45 detects the mounting forces which act on the actuator 20 in the event of a change in the tension within the actuation elements 30. If the actuation elements 30 are tensioned when the slide 27 is moved upward, the mounting forces, for example pressure forces, increase; if the actuation elements 30 are relieved of tension, the mounting forces of the actuator 20 decrease.

FIG. 3 shows two embodiments of the orthopaedic device 1: in the left-hand illustration, the actuator 20 is equipped with a motor 25 as a drive, such that the motor 20 can be activated and deactivated in response to commands from a control device 60 that is connected to the sensor device 40. On the basis of the sensor values and stored control software within the control device 60, the motor 25 is driven in one or the other direction of rotation and is deactivated when the slide 27 has reached the desired position on the spindle 26. The desired position is determined from the detection of the mounting forces by means of the sensor device 40 having the sensor 45, such that the inner periphery of the wall 10 is varied in controlled fashion by way of a measurement of the mounting forces of the actuator 20. The measurement at a virtually arbitrary point on the orthopaedic device 1 allows the contact pressure of the wall 10 against the stump to be precisely adapted for the patient, without the need to actually ascertain the pressure that is exerted between the inner side of the wall 10 and the stump that is received therein. The measurement may be performed at virtually any desired location, because the actuation element 30 can redirect the forces as desired, such that the slide 27 can be displaced along a technically appropriate direction. The actuator 20 with drive, spindle and slide can thus be positioned in a mechanically optimized manner on the orthopaedic device. By way of an optimized mounting of the drive or of the actuation element and an optimized arrangement of the sensor device 40 or of the sensor 45, the mounting forces can be measured with high precision, and it is not necessary to directly measure the pressure between the inner periphery of the wall 10 and the stump or the limb. The mounting forces vary depending on the resistance with which a reduction of the inner periphery of the wall is opposed by the stump or the limb. The greater the resistance, the greater the mounting forces, and the greater the contact pressure of the wall 10 against the limb. The sensor device 40 thus detects a load-dependent displacement of the actuator 20 or of the actuation element 30 or of a diverting device 50, depending on where the sensor 45 or the sensor device 40 is arranged.

In the right-hand illustration of FIG. 3, the position of the slide 27 is adjusted by virtue of the spindle 26 being rotated by way of a manual movement of a hand wheel 24, such that the mounting forces of the drive or actuator 20, as detected by the sensor 45, are not used by the control device to control a motor but are transmitted for example to a display device or to an output device 80, by means of which the magnitude of the mounting forces or the contact pressure of the wall against the stump, as calculated or derived from said mounting forces, is displayed and/or a warning signal is visually and/or acoustically output.

FIG. 4 shows a variant of the invention according to FIG. 1a, illustrating a horizontal section through a prosthesis socket. The wall 10 of the prosthesis socket consists of multiple segments, in this case three segments 11, 12, 14, and the distal end segment 13 may optionally likewise be provided. In addition to the first segment 11 and the separate segment 12, a connection segment 14 is arranged on the first segment 11. The connection segment 14 is coupled to the first segment 11 via a sensor device 40 or via a sensor. Said sensor device 40 detects mounting forces, acting in a peripheral direction, of the connection segment 14 relative to the first segment 11. Furthermore, an end of an actuation element 30 is secured via a lever or a mounting to the connection segment 14. The lever is in turn supported relative to the connection segment 14 via a further sensor 40, and thus detects mounting forces of the actuation element 30 on the wall 10. A further sensor or a further sensor device 40 is situated between the securing point of the actuation element 30 and the actuator 20 in order to detect forces, acting in a radial direction and/or in a peripheral direction, of the actuation element 30 relative to the connection segment 14. A corresponding arrangement is formed, on the opposite side of the connection segment 14, at the first segment 11. A sensor 40 is additionally arranged between the separate, third segment 12 and the actuator 20. By means of all of the sensor devices 40, diverted forces or pressures owing to mounting situations are measured at various points of the wall 10 either by means of pressure sensors or by the detection of derived variables such as changes in length, deformations or the like. Further sensors 70 are also arranged on the inner periphery and outer periphery of the first segment 11, which further sensors can be coupled to the control device 60 of FIG. 3, for example in order to detect myoelectric signals, pressures, oxygen saturation, temperature or accelerations, angular positions, spatial positions, speeds or other movement parameters or state parameters. For example, one of the sensors may be an IMU by means of which movement data of the orthopaedic device can be detected. For example, if intense accelerations are measured, it can be inferred from this that a higher contact pressure of the orthopaedic device against the stump is necessary, and therefore a signal is transmitted from the control device 60 to the drive of the actuator 20. The signal causes an adjustment to be performed automatically until a sufficient displacement of the actuation element 30 has occurred. The extent of the adjustment is determined from the mounting forces which are detected by the sensor devices 40 and which can be assigned to the actuator 20, to the actuation element 30 or to a diverting device 50.

FIG. 5 illustrates various exemplary embodiments of an orthopaedic device, for example a helmet, and an orthotic protective device in the form of a lower leg protector. The helmet has a first wall segment 11 in the shape of a spherical cap, and has a second segment 12 as an inner shell, which first wall segment and second segment are displaceable relative to one another by means of an actuation element 30 in the form of an adjustable head strap, such that the inner periphery of the wall can be varied. In the case of the orthotic protective devices having a shin protector as a first segment 11 and an Achilles tendon protector as a second segment 12 of the wall, a displacement is performed by way of the adjustment strap 30, which is placed around the calf in the region of the ankle condyles. Here, the adjustment strap 30 is the actuation element, which is moved using a hand as a drive for the actuator. For example, the actuator may be a slide, a hand wheel, a grip part of the adjustment strap, a winding apparatus or the like, which is correspondingly displaced. A sensor device 40 is arranged on or in the actuation element 30 in order to detect and record the mounting forces of the actuation element 30 and/or of an actuator 20 (not illustrated). Corresponding actuators and/or sensor devices may also be arranged in the head strap or the lashing element in or on the helmet.

FIG. 6 shows a frontal view of an orthopaedic device 1 in the form of a knee orthosis, in which an orthosis upper part and orthosis lower part are mounted articulatedly on and relative to one another. The upper part is fixed to a thigh, and the lower part is fixed to a lower leg, with the securing to the leg being performed using straps. The fixing of the upper part to the thigh is not illustrated; the fixing to the lower leg is performed by way of a calf fastener, which is at the same time the actuation element. At each of the free ends of the actuation elements, there are arranged fastener devices with which it is possible for the actuation element to be led around the calf and fastened. The lower part of the knee orthosis is thus fixed the lower leg. A contact plate as a segment 12 of the wall of the orthopaedic device is arranged in the region of the shin. The brace-like form of the lower part is the other segment 11 of the wall. On the contact plate 12, there is arranged an adjustment wheel as an actuator 20, behind which there is arranged a sensor for detecting mounting forces of the adjustment wheel 20 relative to the segment 12.

FIGS. 7 and 8 show exemplary embodiments of the arrangement of the sensor device 40 between the contact plate 12 and the actuator 20 in a sectional illustration. The actuator 20 is situated on the frontal outer side of the knee orthosis and is accessible from the outside. The actuator 20 may for example be designed as a turn-lock fastener for tensioning the actuation element 30 and relieving same of tension; the turn-lock fastener may be driven manually or by motor means. The sensor device 40 is situated between the contact plate 12 and the actuator 20, which is supported on the sensor device 40 for example via an intermediate plate or some other support element. Mounting forces may be measured or detected directly or indirectly, for example by means of pressure sensors or by the detection of derived variables such as changes in distance or the like.

FIG. 9 shows an exemplary embodiment of a sensor or of a sensor arrangement 40 in which an actuation element or a second segment 11 of a socket or of a wall is mounted, via pressure springs 35, on a segment 11 of the wall. By means of the pressure springs 35, it is possible to allow a relative displacement between the segments of the wall 11 or between a segment 11 of the wall and an actuation element 30. A permanent magnet 46, which is situated opposite a Hall sensor 45, is arranged on the actuation element 30 or on a segment 11. If the actuation element 30 or a segment 11 of the wall is moved or tensioned by the actuator 20 (not illustrated), the pressure springs 35 are compressed, and the permanent magnet 46 is moved away from the Hall sensor 45. The change in spacing between the Hall sensor 45 and the permanent magnet 46 is measured, and from this, mounting forces between the actuation element 30 and the segment 11 of the wall, or between the segments 11 of the wall 10, are derived and conclusions are drawn regarding the pressure forces or contact pressures acting between the inner wall of an orthopaedic device and the limb or the stump.

1. An orthopaedic device, comprising:
at least one wall which at least partially surrounds a stump or limb when fitted, wherein the wall has a variable inner periphery and forms an entry opening;
an actuator mounted on the orthopaedic device for at least one actuation element which is mounted on the orthopaedic device, wherein the actuator is usable to vary the inner periphery of the at least one wall; and
at least one sensor device is assigned to a mounting of the actuator and/or a mounting of the at least one actuation element, wherein the at least one sensor device determines mounting forces of the actuator and/or of the actuation element.

2. The orthopaedic device as claimed in claim 1, wherein the actuator, the at least one actuation element, and/or a diverting device assigned to the at least one actuation element is mounted in floating fashion on the orthopaedic device, and wherein the at least one sensor device is configured to detect a load-dependent displacement.

3. The orthopaedic device as claimed in claim 1 wherein the at least sensor device has at least one sensor which detects distances, spacings, forces, and/or moments, and wherein the at least one sensor device is designed as a piezo element, capacitive sensor, resistive sensor, inductive displacement transducer, inductive spacing sensor, or optical sensor.

4. The orthopaedic device as claimed in claim 1 wherein the at least one sensor device is designed or arranged to detect forces acting in a proximal-distal direction, in a radial direction, and/or in a peripheral direction of the at least one wall.

5. The orthopaedic device as claimed in claim 1 wherein the at least one the actuation element is designed as a flexible traction element.

6. The orthopaedic device as claimed in claim 1 wherein the actuator comprises a slide, a spindle, or a roller, wherein the slide, the spindle, or the roller is connected to the at least one actuation element.

7. The orthopaedic device as claimed in claim 1 wherein the at least wall is formed in multiple parts or is divided into segments that are displaceable relative to one another.

8. The orthopaedic device as claimed in claim 1 wherein the actuator (20) is driven by a motor or is driven manually.

9. The orthopaedic device as claimed in claim 1 wherein the at least one sensor device is connected to a control device which activates and/or deactivates a motor drive of the actuator based on sensor values and/or the control device transmits a display or output command to a display or output device.

10. The orthopaedic device as claimed in claim 1 wherein the orthopaedic device is designed as a prosthesis socket, an orthosis, or an exoskeleton.

11. A method for controlling an adaptation of an inner periphery of a wall of an orthopaedic device as claimed in claim 1 comprising detecting sensor values by the at least one sensor device, and activating or deactivating a drive of the actuator if set threshold values are overshot and/or undershot.

12. The method as claimed in claim 11, further comprising defining different threshold values for different usage situations, and either automatically identifying a particular usage situation based on the sensor values or manually selecting the particular usage situation.

13. The method as claimed in claim 11 wherein the sensor values are ascertained at different points on the orthopaedic device.

14. The method as claimed in claim 1 further comprising detecting mounting forces of the actuator and/or of the at least one actuation element at mounting points of the actuator and/or of the at least one actuation element in real time and/or irrespective of an operating state of the actuator.
